(19) United States
(12) Patent Application Publication
(10) Pub. No.: US 2017/0355941 A1
(43) Pub. Date: Dec. 14, 2017

(54) CULTURE VESSEL, MEASUREMENT DEVICE, CULTURE METHOD, AND CULTURE DEVICE

(71) Applicant: Hitachi, Ltd., Tokyo (JP)

(72) Inventors: Guangbin ZHOU, Tokyo (JP); Masaharu KIYAMA, Tokyo (JP); Yumiko IGARASHI, Tokyo (JP)

(21) Appl. No.: 15/536,668

(22) PCT Filed: Jan. 30, 2015
(86) PCT No.: PCT/JP2015/052667

§ 371 Date: Jun. 16, 2017

(51) Int. Cl. C12M 3/04; C12M 1/38; G01N 21/41; G01B 11/06; C12M 3/02; C12M 1/09; C12M 1/00; C12M 1/34

(52) U.S. Cl. CPC C12M 3/04; C12M 1/09; C12M 1/38; C12M 1/005; G01N 21/41; G01B 11/06; C12M 3/02; C12M 41/46

(57) ABSTRACT

The present invention provides a culture vessel and a measuring device for measuring a cell sheet thickness nondestructively during culturing. A culture vessel having a lid 4 has a float 8 installed in the interior and the float is structured so as to be uplifted by a culture medium in the culture vessel. Further, a cell sheet thickness measuring device having a sensor head 10 having a light-emitting element 10A and a light-receiving element 10B to detect a positional variation of the float in the vertical direction and a controller 11 to process a signal of the sensor head is configured.

### TECHNICAL FIELD

The present invention relates to cell culture and a technology of measuring the thickness of a cell sheet.

### BACKGROUND ART

Regenerative medicine is drawing attention as an innovative medical treatment for realizing a fundamental remedy of a disabled or deficient cell, tissue, or organ. A regenerative tissue used in regenerative medicine is: produced by exogenously purifying a cell taken from a body of a patient himself or another person and subjecting the cell to processes of amplification, organization, and the like; and transplanted in the body of the patient. Tissue engineering technology is advancing year after year and a method of sheeting cells of a single species and a method of arranging a plurality of cell species sterically and constructing an organ artificially are developed. The regenerative medicine of a cornea and an esophagus using a cell sheet advances in particular. A cell sheet is evaluated by measuring the number of cells in a cell sheet suitable for transplant, a living cell percentage, a thickness, and others before the cell sheet is transplanted and only a cell sheet that has cleared a standard is used for the transplant. In the measurements, the thickness of a cell sheet has been evaluated destructively by selecting a sample from several manufactured cell sheets.

Parent Literature 1 proposes a method of introducing a reference material into a culture medium in a culture vessel and computing the thickness of a cell on the basis of a refraction index.

### CITATION LIST

### Patent Literature

Patent Literature 1: Japanese Unexamined Patent Application Publication No. 2009-106272

### SUMMARY OF INVENTION

### Technical Problem

It has been difficult, however, to apply the method of evaluating the thickness of a cell described in the patent literature to a cell sheet in the confluent state of cells or in the state of stacking cells in layers.

An object of the present invention is to provide a culture vessel, a measuring device, a culture method, and a culture apparatus for solving the above problem.

### Solution to Problem

In order to attain the above object, the present invention provides a culture vessel including a lid, a sidewall, a culture plane, and a float installed in the interior and capable of being uplifted by a culture medium over the culture plane.

Further, in order to attain the above object, the present invention provides a measuring device including a culture vessel including a lid, a sidewall, a culture plane, and a float installed in the interior and capable of being uplifted by a culture medium over the culture plane, a sensor head installed outside the culture vessel and measuring a positional variation of the float in the vertical direction in the interior of the culture vessel, and a controller to process a measurement signal of the sensor head.

Furthermore, in order to attain the above object, the present invention provides a culture method for culturing a cell culture sheet while the positional variation in the vertical direction is measured on the basis of a measurement signal of a sensor head installed outside a culture vessel having a float capable of being uplifted by a culture medium over a culture plane in the interior and measuring a positional variation of the float in the vertical direction in the interior of the culture vessel.

In addition, in order to attain the above object, the present invention provides a culture apparatus including a culture vessel having a float capable of being uplifted by a culture medium over a culture plane in the interior, a sensor head installed outside the culture vessel and measuring a positional variation of the float in the vertical direction in the interior of the culture vessel, a controller to process a measurement signal of the sensor head, a culture medium exchange unit to discharge/inject the culture medium to the culture vessel, a mixed gas exchange unit to discharge/inject a mixed gas to the culture vessel, and a control section to control the controller, the culture medium exchange unit, and the mixed gas exchange unit.

### Advantageous Effects of Invention

The present invention makes it possible to measure the thickness of a cell sheet nondestructively during culturing.

### BRIEF DESCRIPTION OF DRAWINGS

FIG. 1 is a view showing a configuration example of a culture vessel according to a first embodiment.

FIG. 2 is a view showing the principle for measuring the thickness of a cell sheet according to the first embodiment.

FIG. 3 is a view showing a process flow example of a cell sheet thickness measuring device according to the first embodiment.

FIG. 4 is a view showing another configuration example of a culture vessel according to the first embodiment.

FIG. 5 is a view showing another configuration example of a culture vessel according to the first embodiment.

FIG. 6 is a view showing another configuration example of a culture vessel according to the first embodiment.

FIG. 7 is a view showing a configuration example of a culture vessel base according to a second embodiment.

FIG. 8 is a view showing a configuration example of a sensor head transfer mechanism according to the second embodiment.

FIG. 9 is a view showing a schematic configuration example of a culture apparatus according to a third embodiment.

FIG. 10 is a view showing a control system configuration of a culture apparatus according to the third embodiment.

### DESCRIPTION OF EMBODIMENTS

Various embodiments according to the present invention will be explained hereunder in reference to the drawings. Here, in the drawings related to the embodiments, an identical constituent component is represented by an identical numeral.

### First Embodiment

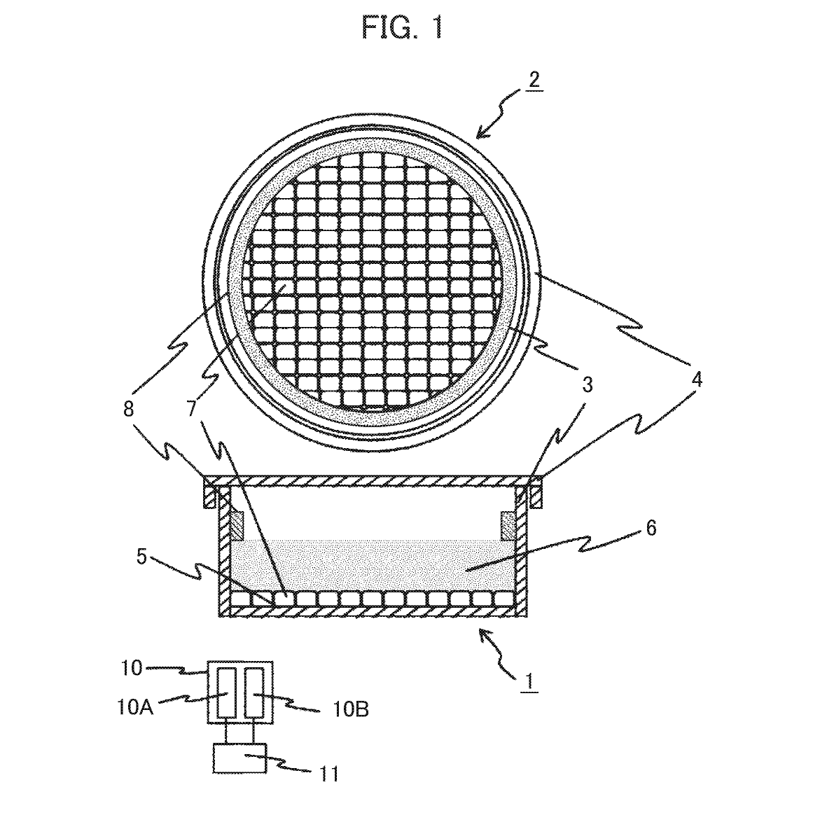

A first embodiment is an embodiment of a cell culture vessel enabling the thickness of a cell sheet to be measured nondestructively during culturing and a cell sheet thickness measuring device using the cell culture vessel. A configuration example of a culture vessel and a measuring device according to the first embodiment is explained in reference to FIG. 1. The present embodiment is: an embodiment of a culture vessel having a float installed in the interior; and also an embodiment of a cell sheet thickness measuring device including a sensor head having a light-emitting element and a light-receiving element to detect a positional variation of the float in the vertical direction and a controller to process a signal of the sensor head.

In FIG. 1, 1 is a front sectional view of a culture vessel, 2 is a plan view of the culture vessel, 3 represents a sidewall of the culture vessel, 4 represents a lid of the culture vessel, 5 represents a culture plane of the culture vessel, 6 represents a culture medium, 7 represents cells, 8 represents a float of a floating-ring type, 10 represents a sensor head, 10A represents a light-emitting element, 10B represents a light-receiving element, and 11 represents a controller functioning as a cell sheet thickness measuring device.

A culture vessel according to the present embodiment includes a sidewall 3 of the culture vessel, a lid 4 of the culture vessel, a culture plane 5 of the culture vessel, and a float 8 installed in the interior. The float 8 is structured so as to be uplifted by a culture medium in the culture vessel, and has a mark to measure a positional variation in the vertical direction in the culture vessel from a culture vessel exterior, namely a reference section for position detection. The mark or the reference section for position detection can be configured, for example, by containing a pigment in a resin used as a float and using the bottom plane, or by pigmenting the bottom plane of a float.

A culture medium 6 is injected into a culture space of the culture vessel and cells 7 are cultured. The floating-ring-type float 8 is arranged in the culture vessel and can be uplifted by the culture medium 6. The float 8 includes a resin or the like similar to the culture vessel as it will be described later. A cell sheet thickness measuring device according to the present embodiment includes the culture vessel and further a sensor head 10 and a controller 11. The sensor head 10 includes a light-emitting element 10A and a light-receiving element 10B. The sensor head 10 has the light-emitting element 10A to irradiate the float 8 with light and the light-receiving element 10B to receive secondary light such as reflected light coming from the float. Irradiation rays are emitted from the light-emitting element 10A and reflected rays reflected by hitting the float 8 can be detected by the light-receiving element 10B. When the vertical position of the float 8 varies, the angle of reflected rays 9B detected by the light-receiving element 10B varies and hence the positional variation of the float 8 in the vertical direction can be identified by the variation of the angle.

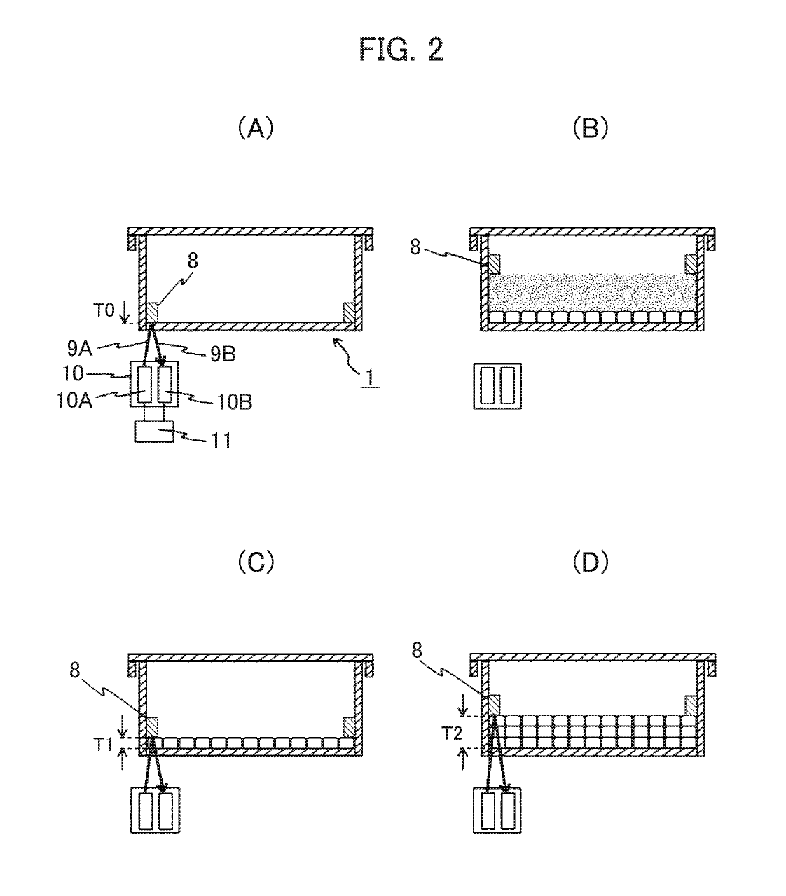

A principle for measuring a cell sheet thickness in cell sheet culture according to the present embodiment is explained sequentially hereunder in reference to FIG. 2.

(A) in FIG. 2 is a view representing a state before a cell is disseminated according to the embodiment shown in FIG. 1. The position of a float 8 in the state where both no culture medium and no cell exist is measured as T0. Successively, as shown in (B) of FIG. 2, cells 7 and a culture medium 6 are injected into a culture vessel and the cells are cultured. The cells come to be in a confluent state and a cell sheet is formed. In the state where the culture medium exists, the float 8 is uplifted by the buoyant force of the culture medium. (C) in FIG. 2 shows a view in the state after the culture medium is discharged. After the culture medium is discharged, a state where the bottom plane of the float 8 touches the top plane of the cell sheet is obtained. A vertical position of the float 8 in the state is measured as T1 and the difference between T1 and T0 measured at (A) in FIG. 2 equals to the thickness of the cell sheet. Further, as shown in (D) of FIG. 2, when the culture continues, the thickness of the cell sheet increases and resultantly the vertical position T2 of the float 8 increases. In other words, by using a culture vessel and a measuring device configured according to the present embodiment, it is possible to measure the thickness of a cell sheet by the variation of the vertical position T of a float 8 during culturing.

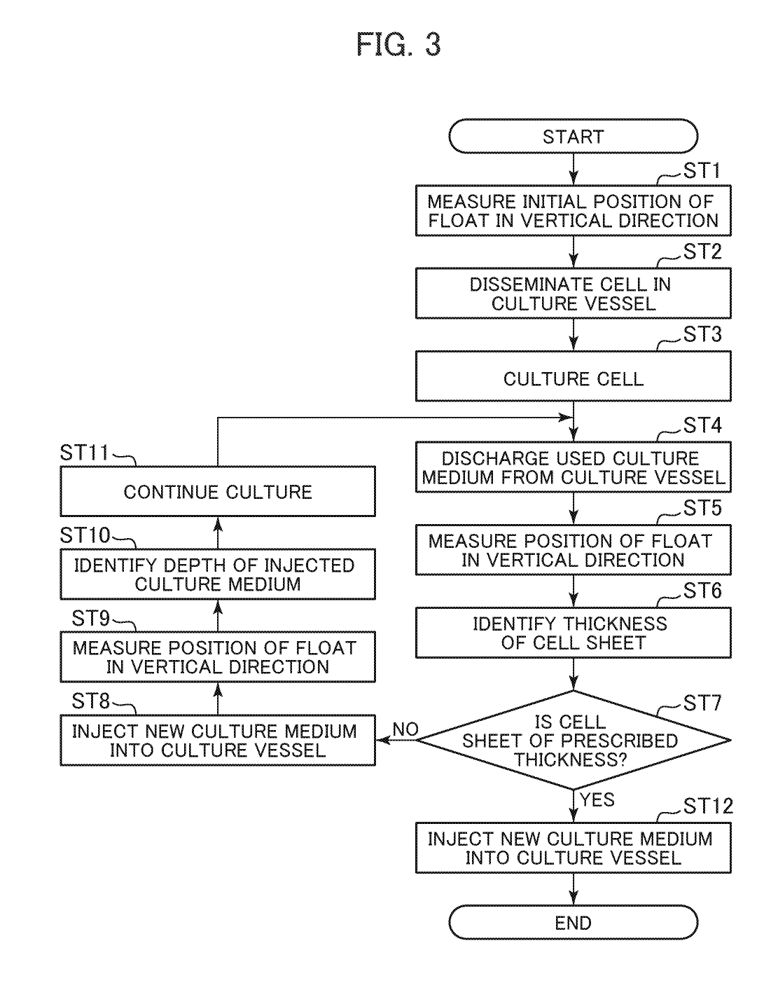

FIG. 3 is a flowchart showing an example of a method of measuring a cell sheet thickness in cell sheet culture by using a culture vessel and a measuring device according to the present embodiment. Here, the processing in the flowchart is carried out by software operated by a controller described earlier or a control terminal that will be explained later. In the figure, when a cultivation process starts, an initial position of a float in the vertical direction is measured before cell dissemination (ST1). Successively, a suspension of cells is injected into the culture vessel and the cells are disseminated (ST2) and then the cells are cultured by adjusting conditions such as temperature and humidity (ST3).

Successively, when prescribed culture medium exchange time comes, the used culture medium in the culture vessel is discharged from the culture vessel (ST4). The float touches a cell top plane thereby and the position of the float in the vertical direction is measured (ST5). The thickness of the cell sheet is identified from the measurement difference between ST5 and ST1 (ST6). Then whether or not the thickness has reached a prescribed cell sheet thickness is judged (ST7), a new culture medium is injected into the culture vessel if the thickness does not reach the prescribed value (ST8), and the position of the float in the vertical direction is measured (ST9). The depth of the injected culture medium is identified from the measurement difference between ST9 and ST5 (ST10) and the culture is continued (ST11). As a result, by the configuration according to the present embodiment of measuring the position of a float in the vertical direction, it is possible not only to measure the thickness of a cultured cell sheet but also to appropriately measure the depth of an injected culture medium, and hence it is possible to culture a cell sheet efficiently by adjusting the quantity of an injected culture medium. By carrying out the steps ST4 to ST11 described above in sequence, a cell sheet is cultured to a prescribed thickness. After the thickness of the cell sheet is confirmed at ST7 to have reached a prescribed thickness, a new culture medium is injected into the culture vessel (ST12) and the culture is finished.

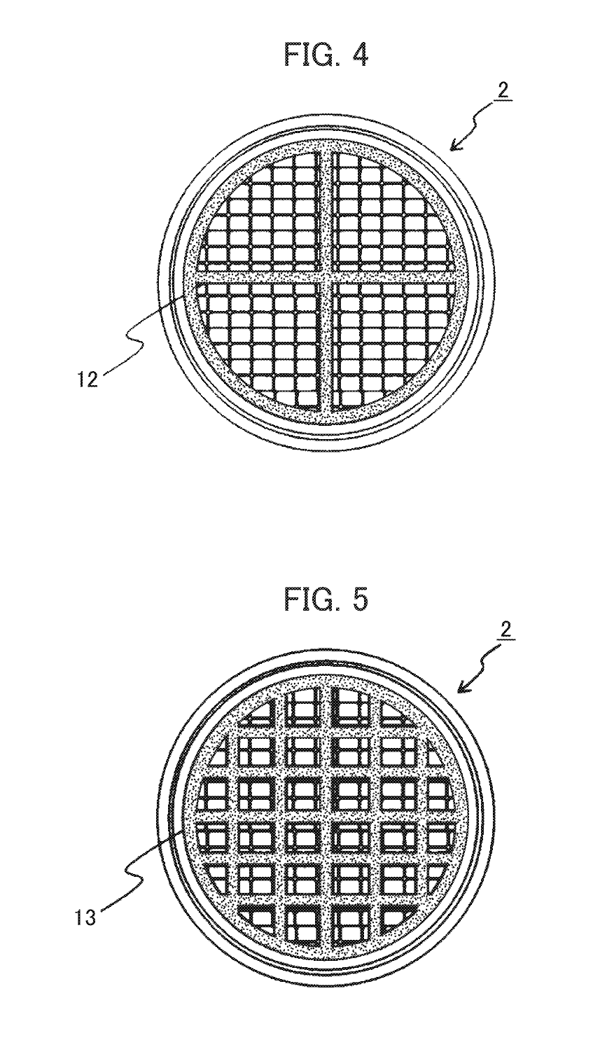

FIGS. 4 and 5 are views showing modifications of a float used in the present embodiment. FIG. 4 shows an example of a cruciform float 12 and FIG. 5 shows an example of a net-type float 13. By using a float of such a shape, it is possible to measure not only the thickness of the outer circumference of a cell sheet but also a thickness distribution over the whole plane including the center of a culture vessel.

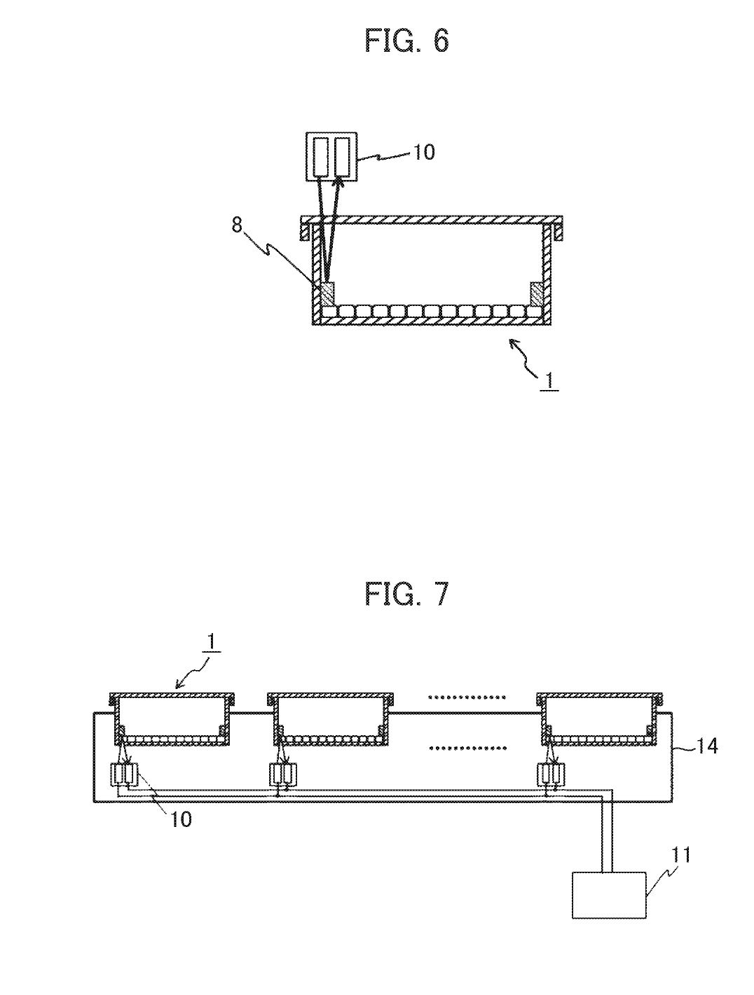

FIG. 6 is a view showing a modification of the installation position of a sensor head used in the present embodiment and is a configuration diagram of installing a sensor heat 10 above a culture vessel. The thickness of a cell sheet can be measured similarly to the above case even when a sensor head 10 is installed above a culture vessel. Here, it goes without saying that similar measurement can be carried out even when a sensor head 10 is installed at a side of a culture vessel.

In a cell sheet thickness measuring device according to the present embodiment, a material having a lower specific gravity than a culture medium is desirably used for a float member. When polystyrene (PS) or polycarbonate (PC) which is used frequently for cell culture is used for example, a float is formed desirably so as to have a hollow structure or a foamed structure in order to make the specific gravity smaller than the specific gravity 1 of a culture medium. Further, a material such as a resin having a high moisture resistance and being compatible with sterilization treatment is desirably used for a culture vessel member and a float member stated above. As a result, the cell sheet thickness measuring device can be compatible with sterilization treatment such as a γ ray treatment, and applicable to physical and chemical applications and regenerative medicine applications.

When a laser beam is used in a sensor head according to the present embodiment, pulse irradiation of the laser beam having a wavelength of 350 to 1,000 nm is desirable in order to reduce the influence on a cell to be cultured.

Meanwhile, although light such as laser beam is used for detecting the vertical position of a float in the present embodiment, the principle of a magnetic field, an electric field, or a contact type is also applicable. Further, although fixed-point measurement of a sensor head is exemplified, measurement at an arbitrary point of a cell sheet can be carried out by installing a sensor head having a movable mechanism. Furthermore, although a round culture dish is exemplified in the present embodiment, a culture dish of another shape such as a square is also applicable by the same principle. Moreover, although single-layered culture is exemplified in the present embodiment, a culture vessel of double-layered culture is also applicable by the same principle.

By a culture vessel and a measuring device according to the first embodiment described above in detail, it is possible to measure a cell sheet thickness nondestructively during culturing.

### Second Embodiment

As a second embodiment, an embodiment of a measuring device to simultaneously measure cell sheet thicknesses in a culture vessel base in which a plurality of cell culture vessels are installed is explained.

FIG. 7 is a view showing a configuration example of a culture vessel base according to the second embodiment. In the figure, sections such as head sensors 10 other than a culture vessel base 14 have the configurations similar to the first embodiment and hence the explanations are omitted here. A sensor head for measuring a cell sheet thickness according to the present embodiment is structured so as to be embedded into a culture vessel base 14. A controller 11 to process a sensor signal singly processes signals of a plurality of sensor heads 10 and the thicknesses of cell sheets in respective culture vessels can therefore be measured nearly simultaneously. That is, the controller 11 controls sensor heads so as to simultaneously measure positional variations of floats in the vertical direction in a plurality of culture vessels.

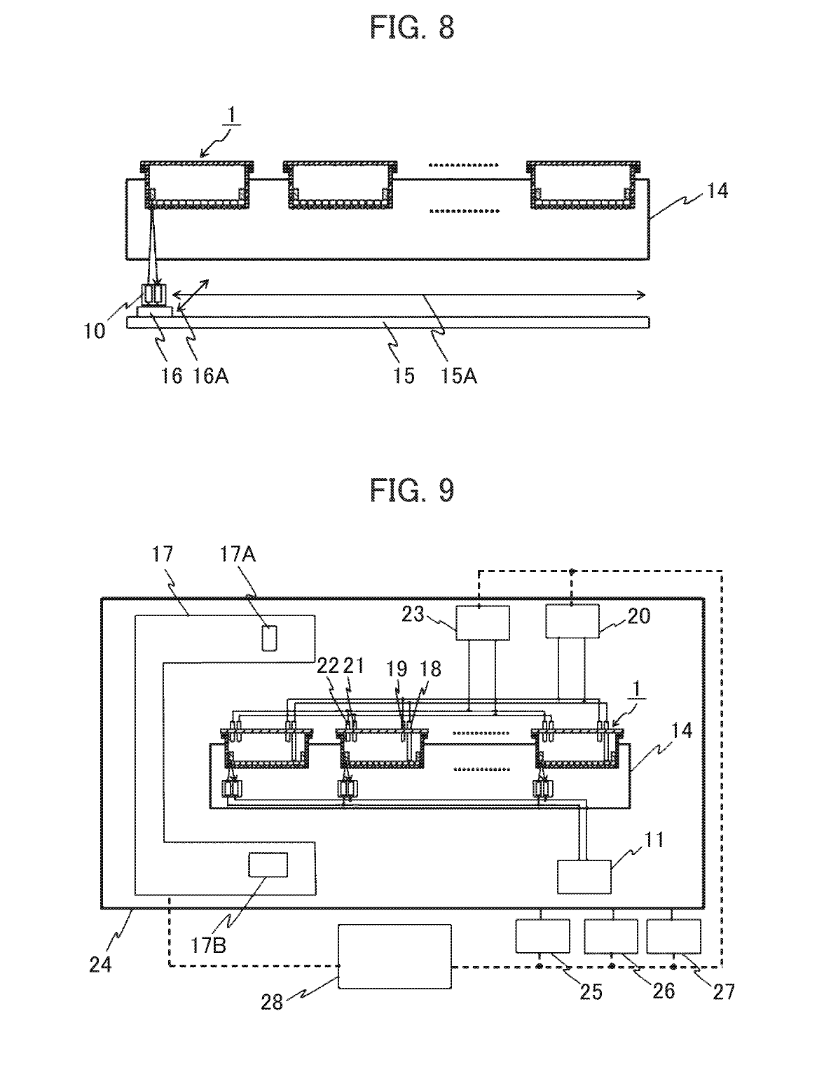

FIG. 8 is a view showing another configuration example of a culture vessel base according to the second embodiment. In the figure, 15 represents a sensor head X-axis transfer mechanism, 15A represents the transfer directions of the sensor head X-axis transfer mechanism, 16 represents a sensor head Y-axis transfer mechanism, and 16A represents the transfer directions of the sensor head Y-axis transfer mechanism. A sensor head 10 for measuring a cell sheet thickness is mounted over the sensor head X-axis transfer mechanism 15 and the sensor head Y-axis transfer mechanism 16, moves to arbitrary positions over the bottom planes of a plurality of culture vessels, and can measure the thicknesses of cell sheets in the culture vessels. That is, a plurality of culture vessels and transfer mechanisms to shift one sensor head to respective positions of the culture vessels are arranged and a controller controls the transfer mechanisms so as to sequentially measure positional variations of floats in the vertical direction in the culture vessels. Here, the transfer mechanisms may be installed above the culture vessels by the same principle.

According to the second embodiment, it is possible to measure cell sheet thicknesses nondestructively while a plurality of cell sheets are cultured simultaneously.

### Third Embodiment

A third embodiment is an embodiment of a culture method and a culture apparatus of a cell sheet by using a culture vessel and a cell sheet thickness measuring device in the respective embodiments explained above. That is, the present embodiment is an embodiment of a culture method and a culture apparatus for exchanging a culture medium and culturing a cell sheet by using a culture medium exchange unit to discharge/inject a culture medium to the aforementioned culture vessel and a mixed gas exchange unit to discharge/inject a mixed gas; and an embodiment of an automatic culture apparatus including a culture vessel having a float capable of being uplifted by a culture medium over a culture plane in the interior, a sensor head installed outside the culture vessel and measuring a positional variation of the float in the vertical direction in the interior of the culture vessel, a controller to process a measurement signal of the sensor head, a culture medium exchange unit to discharge/inject a culture medium to the culture vessel, a mixed gas exchange unit to discharge/inject a mixed gas to the culture vessel, and a control section to control the controller, the culture medium exchange unit, and the mixed gas exchange unit and culturing a cell sheet while the culture medium is exchanged.

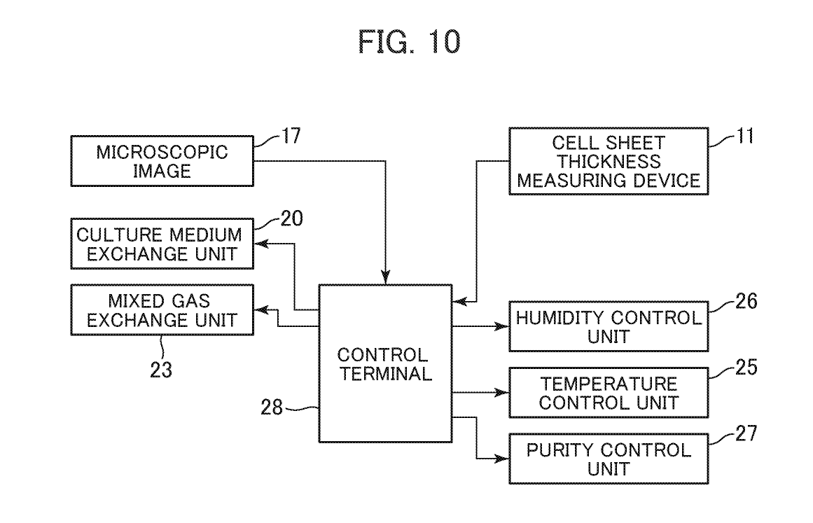

FIG. 9 is a view showing an example of a general schematic configuration of an automatic cell culture apparatus according to the present embodiment. In the culture apparatus shown in FIG. 9, 17 represents a microscope, 17A represents an observation light source, 17B represents a lens, 18 represents a culture medium discharge port of a culture vessel, 19 represents a culture medium injection port of a culture vessel, 20 represents a culture medium exchange unit, 21 represents a discharge port of a mixed gas, 22 represents a supply port of a mixed gas, 23 represents a mixed gas exchange unit, 24 represents an incubator, 25 represents a temperature control unit, 26 represents a humidity control unit, 27 represents a purity control unit, and 28 represents a control terminal as a control section to control respective units. Here, although a controller 11 is shown as another construction controlled by the control terminal 28 in FIGS. 9 and 10, the controller 11 can be a control section configured integrally with the control terminal 28.

FIG. 10 is a block diagram showing a configuration example of a control system of a culture apparatus according to the present embodiment. A culture medium exchange unit 20, a mixed gas exchange unit 23, a temperature control unit 25, a humidity control unit 26, and a purity control unit 27, all of which have been explained earlier, are controlled by a control terminal 28 using a personal computer (PC) including an ordinary computer having a central processing unit (CPU), a memory section, an input/output interface section, a network interface section, and others. The substantial part of the processing flow of cell culture by the control terminal 28 as a control section coincides with the flow of measuring a cell sheet thickness explained in FIG. 3. The control terminal 28 controls the culture of a cell sheet, the measurement of a thickness, and others by implementing software for control stored in the memory section beforehand. Then, the data of a cell sheet thickness measured by a controller 11 functioning as a cell sheet thickness measuring device is inputted to the control terminal 28 from the input/output interface section not shown in the figure and whether or not a cell sheet thickness has reached a prescribed thickness is judged at the control terminal 28 as explained at ST7 in FIG. 3. Further, a microscopic image of a cultured cell is inputted from a microscope 17, transmitted to the control terminal 28 or an exterior server through a network interface section, and processed by various kinds of software for image processing.

A culture apparatus according to the present embodiment can control more uniform culture quality by arranging the mechanisms of automatic cell dissemination, automatic culture medium exchange, automatic observation, and others as well as can culture a cell while a cell sheet thickness is measured nondestructively in the same manner as the first and second embodiments described earlier.

Although various embodiments according to the present invention have heretofore been explained, the present invention is not limited to the above embodiments as long as the feature of the present invention is not hindered and includes various other modifications that can be thought within the range of the technological thought of the present invention. For example, the above embodiments are explained in detail for better understanding the present invention and the present invention is not necessarily limited to the cases having all the configurations explained above. Further, it is possible to replace a part of a configuration of an embodiment with a configuration of another embodiment and add a configuration of an embodiment to a configuration of another embodiment. Furthermore, it is possible to add, delete, or replace another configuration to, from, or with a part of a configuration of an embodiment.

Further, although the configurations, the function, the control terminal, and others that are described above have been explained on the basis of the case of using software for achieving some or all of them, it goes without saying that some or all of them may also be achieved by hardware, for example by designing an integrated circuit.

### LIST OF REFERENCE SIGNS

1. Front sectional view of culture vessel

2. Plan view of culture vessel

3. Sidewall of culture vessel

4. Lid of culture vessel

5. Culture plane of culture vessel

6. Culture medium

7. Cell

8. Float of floating-ring type

9A Irradiation rays

9B Reflected rays

10. Sensor head

11. Controller

12. Cruciform float

13. Net-type float

14. Culture vessel base

15. Sensor head X-axis transfer mechanism

16. Sensor head Y-axis transfer mechanism

17. Microscope

18. Discharge port of culture vessel

19. Injection port of culture vessel

20. Culture medium exchange unit

21. Discharge port of mixed gas

22. Supply port of mixed gas

23. Mixed gas exchange unit

24. Incubator

25. Temperature control unit

26. Humidity control unit

27. Purity control unit

28. Control terminal

1. A culture vessel comprising a lid, a sidewall, a culture plane, and a float installed in the interior and capable of being uplifted by a culture medium over the culture plane.

2. The culture vessel according to claim 1,
wherein the float has a mark to measure a positional variation in the vertical direction from the exterior of the culture vessel.

3. The culture vessel according to claim 1,
wherein the float has a hollow structure or a foamed structure.

4. A measuring device comprising:
a culture vessel including a lid, a sidewall, a culture plane, and a float installed in the interior and capable of being uplifted by a culture medium over the culture plane;
a sensor head installed outside the culture vessel and measuring a positional variation of the float in the vertical direction in the interior of the culture vessel; and
a controller to process a measurement signal of the sensor head.

5. The measuring device according to claim 4,
wherein the float has a mark to measure the positional variation in the vertical direction by the sensor head.

6. The measuring device according to claim 4,
wherein the float has a hollow structure or a foamed structure.

7. The measuring device according to claim 4,
wherein the sensor head has a light-emitting element to irradiate the float with a laser beam and a light-receiving element to receive reflected laser beam from the float.

8. The measuring device according to claim 4,
wherein the measuring device has a plurality of the culture vessels and a plurality of the sensor heads corresponding to the respective culture vessels, and
the controller controls simultaneous measurement of positional variations of the floats in the vertical direction.

9. The measuring device according to claim 4,
wherein the measuring device comprises a plurality of the culture vessels, one the sensor head, and a transfer mechanism to transfer the sensor head to the positions of respective the culture vessels, and
the controller controls the transfer mechanism so as to sequentially measure positional variations of the floats in the vertical direction.

10. A culture method of a cell culture sheet for culturing a cell culture sheet while the positional variation in the vertical direction is measured on the basis of a measurement signal of a sensor head installed outside a culture vessel having a float capable of being uplifted by a culture medium over a culture plane in the interior and measuring a positional variation of the float in the vertical direction in the interior of the culture vessel.

11. The culture method according to claim 10,
wherein the float has a mark to measure the positional variation in the vertical direction by the sensor head.

12. The culture method according to claim 10,
wherein the float has a hollow structure or a foamed structure.

13. A culture apparatus comprising:
a culture vessel having a float capable of being uplifted by a culture medium over a culture plane in the interior;
a sensor head installed outside the culture vessel and measuring a positional variation of the float in the vertical direction in the interior of the culture vessel;
a controller to process a measurement signal of the sensor head;
a culture medium exchange unit to discharge/inject the culture medium to the culture vessel;
a mixed gas exchange unit to discharge/inject a mixed gas to the culture vessel; and
a control section to control the controller, the culture medium exchange unit, and the mixed gas exchange unit.

14. The culture apparatus according to claim 13,
wherein the culture apparatus further comprises a microscope to output a microscopic image of a cell cultured in the culture vessel to the control section.

15. The culture apparatus according to claim 13,
wherein the culture apparatus comprises a plurality of the culture vessels and at least one the sensor head, and
the controller sequentially processes the measurement signals of the culture vessels.
